(19) United States
(12) Patent Application Publication
(10) Pub. No.: US 2006/0083704 A1
(43) Pub. Date: Apr. 20, 2006

(54) Hair conditioning composition comprising high internal phase viscosity silicone copolymer emulsions

(76) Inventor: Peter Marte Torgerson, Washington Court House, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/224,262

(22) Filed: Sep. 12, 2005

Related U.S. Application Data
(60) Provisional application No. 60/620,025, filed on Oct. 19, 2004.

(51) Int. Cl. A61K 8/89

(52) U.S. Cl. 424/70.12

(57) ABSTRACT

A hair conditioning composition comprising silicone copolymer emulsions with an internal phase viscosity of greater than about 120×10⁶ mm²/sec and a gel matrix. The composition of the present invention can provide improved conditioning benefits such as smooth feel and reduced friction to both damaged hair and non-damaged hair, while providing other benefits such as slippery and slick feel on wet hair.

### CROSS-REFERENCE TO RELATED APPLICATIONS

This application claims the benefit of U.S. Provisional Application No. 60/620,025 filed on Oct. 19, 2004, the disclosure of which is incorporated herein in its entirety by reference.

### FIELD

The present invention relates to a hair conditioning composition comprising silicone copolymer emulsions with an internal phase viscosity of greater than about 120×10⁶ mm²/sec and a gel matrix. The composition of the present invention can provide improved conditioning benefits such as smooth feel and reduced friction to both damaged hair and non-damaged hair, while providing other benefits such as slippery and slick feel on wet hair.

### BACKGROUND

Human hair becomes soiled due to its contact with the surrounding environment and from the sebum secreted by the scalp. The soiling of hair causes it to have a dirty feel and an unattractive appearance. The soiling of the hair necessitates shampooing with frequent regularity.

Shampooing cleans the hair by removing excess soil and sebum. However, shampooing can leave the hair in a wet, tangled, and generally unmanageable state. Once the hair dries, it is often left in a dry, rough, lusterless, or frizzy condition due to removal of the hair's natural oils and other natural conditioning and moisturizing components. The hair can further be left with increased levels of static upon drying, which can interfere with combing and result in a condition commonly referred to as “fly-away hair”, or contribute to an undesirable phenomena of “split ends”, particularly for long hair.

A variety of approaches have been developed to condition the hair. A common method of providing conditioning benefit to the hair is through the use of hair conditioning agents such as cationic surfactants and polymers, high melting point fatty compounds, low melting point oils, and silicone compounds. Most of these conditioning agents are known to provide conditioning benefits by depositing on the hair.

Human hair becomes damaged due to, for example, shampooing, combing, permanent waves, and/or coloring the hair. Such damaged hair is often left hydrophilic and/or in a rough condition especially when the hair dries, compared to non-damaged or less damaged hair. There is a need for hair conditioning compositions which provide improved conditioning benefits such as smooth feel and reduced friction on dry hair, especially on damaged hair.

Based on the foregoing, there remains a desire for hair conditioning compositions which provide improved conditioning benefits such as smooth feel and reduced friction on dry hair, especially on damaged hair. There also exists a desire for hair conditioning compositions which provide the above conditioning benefits, while providing other conditioning benefits such as slippery feel and slick feel on wet hair.

### SUMMARY

The present invention is directed to a hair conditioning composition comprising silicone copolymer emulsions with an internal phase viscosity of greater than about 120×10⁶ mm²/sec; and a gel matrix comprising: a cationic surfactant; a high melting point fatty compound; and an aqueous carrier. The hair conditioning composition of the present invention can provide improved conditioning benefits such as smooth feel and reduced friction to both damaged hair and non-damaged hair, while providing other benefits such as slippery and slick feel on wet hair.

### DETAILED DESCRIPTION

The essential components of the personal care composition are described below. Also included is a nonexclusive description of various optional and preferred components useful in embodiments of the present invention. While the specification concludes with claims that particularly point out and distinctly claim the invention, it is believed the present invention will be better understood from the following description.

All percentages, parts and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified. The term “weight percent” may be denoted as “wt. %” herein.

All molecular weights as used herein are weight average molecular weights expressed as grams/mole, unless otherwise specified.

The compositions and methods/processes of the present invention can comprise, consist of, and consist essentially of the essential elements and limitations of the invention described herein, as well as any of the additional or optional ingredients, components, steps, or limitations described herein.

Herein, “mixtures” is meant to include a simple combination of materials and any compounds that may result from their combination.

The hair conditioning composition of the present invention comprises a silicone copolymer emulsion and a gel matrix. The composition is prepared by a method comprising the step of mixing the silicone copolymer emulsion with the gel matrix.

Damaged hair is less hydrophobic compared to non-damaged and/or less damaged hair. It is believed that by providing improved hydrophobicity to hair, the hair conditioning composition can provide improved smooth feel and reduced friction to the hair. It is also believed that the improved hydrophobicity to the hair can be provided by some other preferred features of the present invention, for example, the use of additional materials such as other silicones, hydrocarbons, and/or cationic surfactants. Further, without being limited to the theory, it is believed that improved hydrophobicity provides improved tolerance to the hair for humidity in the surrounding circumstances, and thus provides reduced frizziness and/or fly-aways on rainy and/or humid days.

The hair conditioning composition of the present invention is preferably substantially free of anionic compounds. Anionic compounds herein include anionic surfactants and anionic polymers. In the present invention, “substantially free of anionic compounds” means that the composition contains 1% or less, preferably 0.5% or less, more preferably less than 0.01% of anionic compounds.

The hair conditioning composition of the present invention has a pH of preferably from about 2 to about 9, more preferably from about 3 to about 7.

A. Silicone Copolymer Emulsion

The silicone copolymer emulsion provides improved conditioning benefits such as smooth feel and reduced friction. The silicone copolymer emulsion is present in an amount of from about 0.1% to about 15%, preferably from about 0.5% to about 10%, more preferably from about 1% to about 8% by weight of the composition.

The silicone copolymer emulsion has an internal phase viscosity at 25° C. of greater than about 120×10⁶ mm²/sec, preferably greater than about 150×10⁶ mm²/sec. To measure the internal phase viscosity of the silicone copolymer emulsion, one must first break the polymer from the emulsion. By way of example, the following procedure can be used to break the polymer from the emulsion: 1) add 10 grams of an emulsion sample to 15 milliliters of isopropyl alcohol; 2) mix well with a spatula; 3) decant the isopropyl alcohol; 4) add 10 milliliters of acetone and knead polymer with spatula; 5) decant the acetone; 6) place polymer in an aluminum container and flatten/dry with a paper towel; and 7) dry for two hours in an 80° C. The polymer can then be tested using any known rheometer, such as, for example, a CarriMed, Haake, or Monsanto rheometer, which operates in the dynamic shear mode. The internal phase viscosity values can be obtained by recording the dynamic viscosity (n′) at a 9.900*10⁻³ Hz frequency point.

The average particle size of the emulsions is preferably less than about 1 micron, more preferably less than about 0.7 micron. The silicone copolymer emulsions of the present invention comprise a silicone copolymer, at least one surfactant, and water.

The silicone copolymer results from the addition reaction of the following two materials in the presence of a metal containing catalyst:
(a) a polysiloxane with reactive groups on both termini, represented by formula (I) 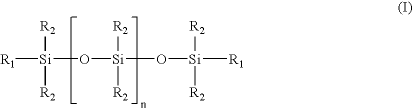
   wherein:

R₁ is a group capable of reacting by chain addition reaction such as, for example, a hydrogen atom, an aliphatic group with ethylenic unsaturation (i.e. vinyl, allyl, or hexenyl), a hydroxyl group, an alkoxyl group (i.e. methoxy, ethoxy, or propoxy), an acetoxyl group, or an amino or alkylamino group; preferably, R₁ is hydrogen or an aliphatic group with ethylenic unsaturation; more preferably, R₁ is hydrogen;

R₂ is alkyl, cycloalkyl, aryl, or alkylaryl and may include additional functional groups such as ethers, hydroxyls, amines, carboxyls, thiols esters, and sulfonates; preferably, R₂ is methyl. Optionally, a small mole percentage of the R₂ groups may be reactive groups as described above for R₁, to produce a polymer which is substantially linear but with a small amount of branching. In this case, preferably the level of R₂ groups equivalent to R₁ groups is less than about 10% on a mole percentage basis, and more preferably less than about 2%;

n is a whole number such that the polysiloxane of formula (I) has a viscosity of from about 1 mm²/sec to about 1×10⁶ mm²/sec; and,

(b) at least one silicone compound or non-silicone compound comprising at least one or at most two groups capable of reacting with the R₁ groups of the polysiloxane in formula (I); preferably, the reactive group is an aliphatic group with ethylenic unsaturation.

The metal containing catalysts used in the above described reactions are often specific to the particular reaction. Such catalysts are known in the art. Generally, they are materials containing metals such as platinum, rhodium, tin, titanium, copper, lead, etc.

The mixture used to form the emulsion also contains at least one surfactant. This can include non-ionic surfactants, cationic surfactants, anionic surfactants, alkylpolysaccharides, amphoteric surfactants, and the like. The above surfactants can be used individually or in combination.

The method of making the silicone copolymer emulsions described herein comprises the steps of 1) mixing materials (a) described above with material (b) described above, followed by mixing in an appropriate metal containing catalyst, such that material (b) is capable of reacting with material (a) in the presence of the metal containing catalyst; 2) further mixing in at least one surfactant and water; and 3) emulsifying the mixture. Methods of making such silicone copolymer emulsions are disclosed in U.S. Pat. No. 6,013,682; WO01/58986 A1; and EP0874017 A2.

B. Gel Matrix

Compositions of the present invention comprise a gel matrix comprising a cationic surfactant, a high melting fatty compound, and an aqueous carrier. The cationic surfactant, together with the high melting fatty compound, and an aqueous carrier, provides a gel matrix which is suitable for providing various conditioning benefits, especially slippery and slick feel on wet hair. In view of providing the above gel matrix, the cationic surfactant and the high melting point fatty compound are contained at a level such that the mole ratio of the cationic surfactant to the high melting point fatty compound is in the range of, preferably from about 1:1 to about 1:10, more preferably from about 1:2 to about 1:6, in view of providing the above conditioning benefits especially slippery and slick feel on wet hair.

1. Cationic Surfactant

The compositions of the present invention comprise a cationic surfactant. A variety of cationic surfactants including mono- and di-alkyl chain cationic surfactants can be used in the compositions of the present invention as described below. Among them, preferred are mono-alkyl chain cationic surfactants such as mono-alkyl chain quaternary ammonium salts. The mono-alkyl chain quaternary ammonium salts useful herein are those having mono-long alkyl chain which has from 16 to 30 carbon atoms, preferably from 16 to 22 carbon atoms. Highly preferred mono-alkyl chain quaternary ammonium salts are, for example, cetyl trimethyl ammonium chloride, stearyl trimethyl ammonium chloride, behenyl trimethyl ammonium chloride. Although the mono-alkyl chain cationic surfactants are preferred, other cationic surfactants such as di-alkyl chain cationic surfactants may also be used alone, or in combination with the mono-alkyl chain cationic surfactants and/or nonionic surfactants.

Cationic surfactants useful herein include, for example, those corresponding to the general formula (I): 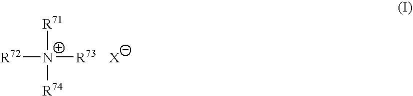
wherein at least one of R⁷¹, R⁷², R⁷³ and R⁷⁴ is selected from an aliphatic group of from 16 to 30 carbon atoms that optionally includes an aromatic, alkoxy, polyoxyalkylene, alkylamido, hydroxyalkyl, aryl or alkylaryl group having up to about 30 carbon atoms, the remainder of R⁷¹, R⁷², R⁷³ and R⁷⁴ are independently selected from a group consisting of a hydrogen, an aliphatic group of from 1 to about 22 carbon atoms, and an aromatic, alkoxy, polyoxyalkylene, alkylamido, hydroxyalkyl, aryl or alkylaryl group having up to about 22 carbon atoms; and X is a salt-forming anion such as those selected from halogen, (e.g. chloride, bromide), acetate, citrate, lactate, glycolate, phosphate, nitrate, sulfonate, sulfate, glutamate, alkylsulfate, and alkyl sulfonate radicals. The aliphatic groups can contain, in addition to carbon and hydrogen atoms, ether linkages, and other groups such as amino groups. The longer chain aliphatic groups, e.g., those of about 12 carbons, or higher, can be saturated or unsaturated. Preferred is when R⁷¹, R⁷², R⁷³ and R⁷⁴ are independently selected from C₁ to about C₂₂ alkyl.

Among the cationic surfactants of general formula (I), preferred are those containing in the molecule at least one alkyl chain having at least 16 carbons. Nonlimiting examples of such preferred cationic surfactants include: behenyl trimethyl ammonium chloride available, for example, with tradename Genamine KDMP from Clariant, with tradename INCROQUAT TMC-80 from Croda, and with tradename ECONOL TM22 from Sanyo Kasei; cetyl trimethyl ammonium chloride available, for example, with tradename CTAC 30KC from KCI, and with tradename CA-2350 from Nikko Chemicals; stearyl trimethyl ammonium chloride available, for example, with tradename Genamine STACP from Clariant; olealkonium chloride available, for example, with tradename Incroquat O-50 from Croda; hydrogenated tallow alkyl trimethyl ammonium chloride, dialkyl (14-18) dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride.

Also preferred are hydrophilically substituted cationic surfactants in which at least one of the substituents contain one or more aromatic, ether, ester, amido, or amino moieties present as substituents or as linkages in the radical chain, wherein at least one of the R⁷¹-R⁷⁴ radicals contain one or more hydrophilic moieties selected from alkoxy (preferably C₁-C₃ alkoxy), polyoxyalkylene (preferably C₁-C₃ polyoxyalkylene), alkylamido, hydroxyalkyl, alkylester, and combinations thereof. Preferably, the hydrophilically substituted cationic conditioning surfactant contains from 2 to about 10 nonionic hydrophile moieties located within the above stated ranges. Highly preferred hydrophilically substituted cationic surfactants include dialkylamido ethyl hydroxyethylmonium salt, dialkylamidoethyl dimonium salt, dialkyloyl ethyl hydroxyethylmonium salt, dialkyloyl ethyldimonium salt, and mixtures thereof; for example, commercially available under the following tradenames; VARISOFT 110, VARISOFT 222, VARIQUAT K1215 and VARIQUAT 638 from Witco Chemical, MACKPRO KLP, MACKPRO WLW, MACKPRO MLP, MACKPRO NSP, MACKPRO NLW, MACKPRO WWP, MACKPRO NLP, MACKPRO SLP from McIntyre, ETHOQUAD 18/25, ETHOQUAD O/12PG, ETHOQUAD C/25, ETHOQUAD S/25, and ETHODUOQUAD from Akzo, DEHYQUAT SP from Henkel, and ATLAS G265 from ICI Americas. Babassuamidopropalkonium Chloride available from Croda under the tradename Incroquat BA-85 is also preferably used in the composition.

Amines are suitable as cationic surfactants. Primary, secondary, and tertiary fatty amines are useful. Particularly useful are tertiary amido amines having an alkyl group of from about 12 to about 22 carbons. Exemplary tertiary amido amines include: stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide. Useful amines in the present invention are disclosed in U.S. Pat. No. 4,275,055, Nachtigal, et al. These amines can also be used in combination with acids such as l-glutamic acid, lactic acid, hydrochloric acid, malic acid, succinic acid, acetic acid, fumaric acid, tartaric acid, citric acid, l-glutamic hydrochloride, maleic acid, and mixtures thereof; more preferably l-glutamic acid, lactic acid, citric acid. The amines herein are preferably partially neutralized with any of the acids at a molar ratio of the amine to the acid of from about 1: 0.3 to about 1: 2, more preferably from about 1: 0.4 to about 1: 1.

The compositions of the present invention preferably comprise the cationic surfactant in amount of from about 0.1% to about 10%, more preferably from about 1% to about 8%, still more preferably from about 1.5% to about 5% by weight of the composition.

2. High Melting Point Fatty Compound

Compositions of the present invention comprise a high melting point fatty compound. The high melting point fatty compounds useful herein have a melting point of about 25° C. or higher, and are selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than about 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

The high melting point fatty compound can be included in the composition at a level of from about 0.1% to about 20%, preferably from about 1% to about 10%, still more preferably from about 2% to about 8%, by weight of the composition.

The fatty alcohols useful herein are those having from about 14 to about 30 carbon atoms, preferably from about 16 to about 22 carbon atoms. These fatty alcohols are saturated and can be straight or branched chain alcohols. Nonlimiting examples of fatty alcohols include cetyl alcohol, stearyl alcohol, behenyl alcohol, and mixtures thereof.

The fatty acids useful herein are those having from about 10 to about 30 carbon atoms, preferably from about 12 to about 22 carbon atoms, and more preferably from about 16 to about 22 carbon atoms. These fatty acids are saturated and can be straight or branched chain acids. Also included are diacids, triacids, and other multiple acids which meet the requirements herein. Also included herein are salts of these fatty acids. Nonlimiting examples of fatty acids include lauric acid, palmitic acid, stearic acid, behenic acid, sebacic acid, and mixtures thereof.

The fatty alcohol derivatives and fatty acid derivatives useful herein include alkyl ethers of fatty alcohols, alkoxylated fatty alcohols, alkyl ethers of alkoxylated fatty alcohols, esters of fatty alcohols, fatty acid esters of compounds having esterifiable hydroxy groups, hydroxy-substituted fatty acids, and mixtures thereof. Nonlimiting examples of fatty alcohol derivatives and fatty acid derivatives include materials such as methyl stearyl ether; the ceteth series of compounds such as ceteth-1 through ceteth-45, which are ethylene glycol ethers of cetyl alcohol, wherein the numeric designation indicates the number of ethylene glycol moieties present; the steareth series of compounds such as steareth-1 through 10, which are ethylene glycol ethers of steareth alcohol, wherein the numeric designation indicates the number of ethylene glycol moieties present; ceteareth 1 through ceteareth-10, which are the ethylene glycol ethers of ceteareth alcohol, i.e. a mixture of fatty alcohols containing predominantly cetyl and stearyl alcohol, wherein the numeric designation indicates the number of ethylene glycol moieties present; C₁-C₃₀ alkyl ethers of the ceteth, steareth, and ceteareth compounds just described; polyoxyethylene ethers of behenyl alcohol; ethyl stearate, cetyl stearate, cetyl palmitate, stearyl stearate, myristyl myristate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, polyglyceryl stearate, glyceryl monostearate, glyceryl distearate, glyceryl tristearate, and mixtures thereof.

High melting point fatty compounds of a single compound of high purity are preferred. Single compounds of pure fatty alcohols selected from the group of pure cetyl alcohol, stearyl alcohol, and behenyl alcohol are highly preferred. By “pure” herein, what is meant is that the compound has a purity of at least about 90%, preferably at least about 95%. These single compounds of high purity provide good rinsability from the hair when the consumer rinses off the composition.

Commercially available high melting point fatty compounds useful herein include: cetyl alcohol, stearyl alcohol, and behenyl alcohol having tradenames KONOL series available from Shin Nihon Rika (Osaka, Japan), and NAA series available from NOF (Tokyo, Japan); pure behenyl alcohol having tradename 1-DOCOSANOL available from WAKO (Osaka, Japan), various fatty acids having tradenames NEO-FAT available from Akzo (Chicago Ill., USA), HYSTRENE available from Witco Corp. (Dublin Ohio, USA), and DERMA available from Vevy (Genova, Italy).

3. Aqueous Carrier

Compositions of the present invention comprise an aqueous carrier. The level and species of the carrier are selected according to the compatibility with other components, and other desired characteristics of the product.

The carrier useful in the present invention includes water and water solutions of lower alkyl alcohols and polyhydric alcohols. The lower alkyl alcohols useful herein are monohydric alcohols having from about 1 to about 6 carbons, more preferably ethanol and isopropanol. The polyhydric alcohols useful herein include propylene glycol, hexylene glycol, glycerin, and propane diol.

Preferably, the aqueous carrier is substantially water. Deionized water is preferably used. Water from natural sources including mineral cations can also be used, depending on the desired characteristic of the product. Generally, the compositions of the present invention comprise from about 20% to about 95%, preferably from about 30% to about 92%, and more preferably from about 50% to about 90% water.

C. Additional Components

Compositions of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.001% to about 10%, preferably up to about 5% by weight of the composition.

A wide variety of other additional components can be formulated into the present compositions. These include: other conditioning agents such as hydrolysed collagen with tradename Peptein 2000 available from Hormel, vitamin E with tradename Emix-d available from Eisai, panthenol available from Roche, panthenyl ethyl ether available from Roche, hydrolysed keratin, proteins, plant extracts, and nutrients; emollients such as PPG-3 myristyl ether with tradename Varonic APM available from Goldschmidt, Trimethyl pentanol hydroxyethyl ether, PPG-11 stearyl ether with tradename Varonic APS available from Goldschmidt, Stearyl heptanoate with tradename Tegosoft SH available from Goldschmidt, Lactil (mixture of Sodium lactate, Sodium PCA, Glycine, Fructose, Urea, Niacinamide, Inositol, Sodium Benzoate, and Lactic acid) available from Goldschmidt, Ethyl hexyl palmitate with tradename Saracos available from Nishin Seiyu and with tradename Tegosoft OP available from Goldschmidt; hair-fixative polymers such as amphoteric fixative polymers, cationic fixative polymers, anionic fixative polymers, nonionic fixative polymers, and silicone grafted copolymers; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes; hair oxidizing (bleaching) agents, such as hydrogen peroxide, perborate and persulfate salts; hair reducing agents such as the thioglycolates; perfumes; and sequestering agents, such as disodium ethylenediamine tetra-acetate; ultraviolet and infrared screening and absorbing agents such as octyl salicylate; and antidandruff agents such as zinc pyrithione and salicylic acid.

1. Silicone

Compositions of the present invention may further comprise an additional silicone compound. The silicone compound can be included in an amount of from about 0.1% to about 10%, more preferably from about 0.25% to about 8%, still more preferably from about 0.5% to about 3% by weight of the composition.

The silicone compounds hereof can include volatile soluble or insoluble, or nonvolatile soluble or insoluble silicone conditioning agents. By soluble what is meant is that the silicone compound is miscible with the carrier of the composition so as to form part of the same phase. By insoluble what is meant is that the silicone forms a separate, discontinuous phase from the carrier, such as in the form of an emulsion or a suspension of droplets of the silicone. The silicone compounds herein may be made by conventional polymerization, or emulsion polymerization.

The silicone compounds for use herein will preferably have a viscosity of from about 1,000 to about 2,000,000 centistokes at 25° C., more preferably from about 10,000 to about 1,800,000, and even more preferably from about 25,000 to about 1,500,000. The viscosity can be measured by means of a glass capillary viscometer as set forth in Dow Corning Corporate Test Method CTM0004, Jul. 20, 1970, which is incorporated by reference herein in its entirety. Silicone compounds of high molecular weight may be made by emulsion polymerization.

Silicone compounds useful herein include polyalkyl polyaryl siloxanes, polyalkyleneoxide-modified siloxanes, silicone resins, amino-substituted siloxanes, and mixtures thereof. The silicone compound is preferably selected from the group consisting of polyalkyl polyaryl siloxanes, polyalkyleneoxide-modified siloxanes, silicone resins, and mixtures thereof, and more preferably from one or more polyalkyl polyaryl siloxanes.

Polyalkyl polyaryl siloxanes useful here in include those with the following structure (I) 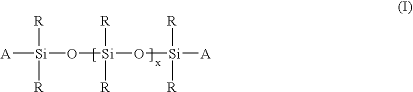
wherein R is alkyl or aryl, and x is an integer from about 7 to about 8,000. “A” represents groups which block the ends of the silicone chains. The alkyl or aryl groups substituted on the siloxane chain (R) or at the ends of the siloxane chains (A) can have any structure as long as the resulting silicone remains fluid at room temperature, is dispersible, is neither irritating, toxic nor otherwise harmful when applied to the hair, is compatible with the other components of the composition, is chemically stable under normal use and storage conditions, and is capable of being deposited on and conditions the hair. Suitable A groups include hydroxy, methyl, methoxy, ethoxy, propoxy, and aryloxy. The two R groups on the silicon atom may represent the same group or different groups. Preferably, the two R groups represent the same group. Suitable R groups include methyl, ethyl, propyl, phenyl, methylphenyl and phenylmethyl. The preferred silicone compounds are polydimethylsiloxane, polydiethylsiloxane, and polymethylphenylsiloxane. Polydimethylsiloxane, which is also known as dimethicone, is especially preferred. The polyalkylsiloxanes that can be used include, for example, polydimethylsiloxanes. These silicone compounds are available, for example, from the General Electric Company in their ViscasilR and SF 96 series, and from Dow Corning in their Dow Corning 200 series. Polymethylphenylsiloxanes, for example, from the General Electric Company as SF 1075 methyl phenyl fluid or from Dow Corning as 556 Cosmetic Grade Fluid, are useful herein.

Also preferred, for enhancing the shine characteristics of hair, are highly arylated silicone compounds, such as highly phenylated polyethyl silicone having refractive index of about 1.46 or higher, especially about 1.52 or higher. When these high refractive index silicone compounds are used, they should be mixed with a spreading agent, such as a surfactant or a silicone resin, as described below to decrease the surface tension and enhance the film forming ability of the material.

Another polyalkyl polyaryl siloxane that can be especially useful is a silicone gum. The term “silicone gum”, as used herein, means a polyorganosiloxane material having a viscosity at 25° C. of greater than or equal to 1,000,000 centistokes. It is recognized that the silicone gums described herein can also have some overlap with the above-disclosed silicone compounds. This overlap is not intended as a limitation on any of these materials. Silicone gums are described by Petrarch, and others including U.S. Pat. No. 4,152,416, to Spitzer et al., issued May 1, 1979 and Noll, Walter, Chemistry and Technology of Silicones, New York: Academic Press 1968. Also describing silicone gums are General Electric Silicone Rubber Product Data Sheets SE 30, SE 33, SE 54 and SE 76. All of these described references are incorporated herein by reference in their entirety. The “silicone gums” will typically have a mass molecular weight in excess of about 200,000, generally between about 200,000 and about 1,000,000. Specific examples include polydimethylsiloxane, poly(dimethylsiloxane methylvinylsiloxane) copolymer, poly(dimethylsiloxane diphenylsiloxane methylvinylsiloxane) copolymer and mixtures thereof.

Polyalkyleneoxide-modified siloxanes useful herein include, for example, polypropylene oxide modified and polyethylene oxide modified polydimethylsiloxane. The ethylene oxide and polypropylene oxide level should be sufficiently low so as not to interfere with the dispersibility characteristics of the silicone. These materials are also known as dimethicone copolyols.

Silicone resins, which are highly crosslinked polymeric siloxane systems, are useful herein. The crosslinking is introduced through the incorporation of tri-functional and tetra-functional silanes with mono-functional or di-functional, or both, silanes during manufacture of the silicone resin. As is well understood in the art, the degree of crosslinking that is required in order to result in a silicone resin will vary according to the specific silane units incorporated into the silicone resin. In general, silicone materials which have a sufficient level of trifunctional and tetrafunctional siloxane monomer units, and hence, a sufficient level of crosslinking, such that they dry down to a rigid, or hard, film are considered to be silicone resins. The ratio of oxygen atoms to silicon atoms is indicative of the level of crosslinking in a particular silicone material. Silicone materials which have at least about 1.1 oxygen atoms per silicon atom will generally be silicone resins herein. Preferably, the ratio of oxygen:silicon atoms is at least about 1.2:1.0. Silanes used in the manufacture of silicone resins include monomethyl-, dimethyl-, trimethyl-, monophenyl-, diphenyl-, methylphenyl-, monovinyl-, and methylvinylchlorosilanes, and tetrachlorosilane, with the methyl substituted silanes being most commonly utilized. Preferred resins are offered by General Electric as GE SS4230 and SS4267. Commercially available silicone resins will generally be supplied in a dissolved form in a low viscosity volatile or nonvolatile silicone fluid. The silicone resins for use herein should be supplied and incorporated into the present compositions in such dissolved form, as will be readily apparent to those skilled in the art. Without being bound by theory, it is believed that the silicone resins can enhance deposition of other silicone compounds on the hair and can enhance the glossiness of hair with high refractive index volumes.

Other useful silicone resins are silicone resin powders such as the material given the CTFA designation polymethylsilsequioxane, which is commercially available as Tospearl™ from Toshiba Silicones.

Silicone resins can conveniently be identified according to a shorthand nomenclature system well known to those skilled in the art as the “MDTQ” nomenclature. Under this system, the silicone is described according to the presence of various siloxane monomer units which make up the silicone. Briefly, the symbol M denotes the mono-functional unit (CH₃)₃SiO_{0.5}; D denotes the difunctional unit (CH₃)₂SiO; T denotes the trifunctional unit (CH₃)SiO_{1.5}; and Q denotes the quadri- or tetra-functional unit SiO₂. Primes of the unit symbols, e.g., M′, D′, T′, and Q′ denote substituents other than methyl, and must be specifically defined for each occurrence. Typical alternate substituents include groups such as vinyl, phenyl, amino, hydroxyl, etc. The molar ratios of the various units, either in terms of subscripts to the symbols indicating the total number of each type of unit in the silicone, or an average thereof, or as specifically indicated ratios in combination with molecular weight, complete the description of the silicone material under the MDTQ system. Higher relative molar amounts of T, Q, T′ and/or Q′ to D, D′, M and/or or M′ in a silicone resin is indicative of higher levels of crosslinking. As discussed before, however, the overall level of crosslinking can also be indicated by the oxygen to silicon ratio.

The silicone resins for use herein which are preferred are MQ, MT, MTQ, MQ and MDTQ resins. Thus, the preferred silicone substituent is methyl. Especially preferred are MQ resins wherein the M:Q ratio is from about 0.5:1.0 to about 1.5:1.0 and the average molecular weight of the resin is from about 1000 to about 10,000.

Amino-substituted siloxanes useful herein include those represented by the following structure (II) 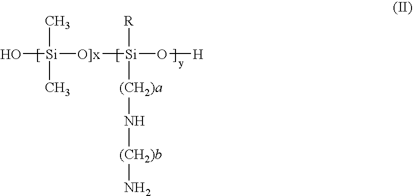
wherein R is CH₃ or OH, x and y are integers which depend on the molecular weight, the average molecular weight being approximately between 5,000 and 10,000; both a and b denote an integer from 2 to 8. This polymer is also known as “amodimethicone”.

Suitable amino-substituted siloxane fluids include those represented by the formula (III)
(R₁)ₐG₃₋ₐ—Si—(—OSiG₂)ₙ—(—OSiG_{b}(R₁)_{2-b})ₘ—O—SiG₃₋ₐ(R₁)ₐ (III)
in which G is chosen from the group consisting of hydrogen, phenyl, OH, C₁-C₈ alkyl and preferably methyl; a denotes 0 or an integer from 1 to 3, and preferably equals 0; b denotes 0 or 1 and preferably equals 1; the sum n+m is a number from 1 to 2,000 and preferably from 50 to 150, n being able to denote a number from 0 to 1,999 and preferably from 49 to 149 and m being able to denote an integer from 1 to 2,000 and preferably from 1 to 10; R₁ is a monovalent radical of formula CqH_{2q}L in which q is an integer from 2 to 8 and L is chosen from the groups
—N(R₂)CH₂—CH₂—N(R₂)₂
—N(R₂)₂
—N(R₂)₃A⁻
—N(R₂)CH₂—CH₂—NR₂H₂A⁻
in which R₂ is chosen from the group consisting of hydrogen, phenyl, benzyl, a saturated hydrocarbon radical, preferably an alkyl radical containing from 1 to 20 carbon atoms, and A⁻ denotes a halide ion.

An especially preferred amino-substituted siloxane corresponding to formula (III) is the polymer known as “trimethylsilylamodimethicone”, of formula (IV):

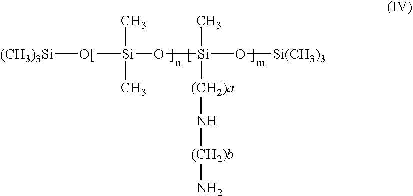

In this formula n and m are selected depending on the molecular weight of the compound desired; both a and b denote an integer from 2 to 8.

Other amino-substituted siloxane which can be used are represented by the formula (V): 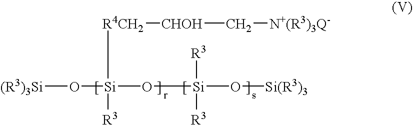
where R³ denotes a monovalent hydrocarbon radical having from 1 to 18 carbon atoms, preferably an alkyl or alkenyl radical such as methyl; R⁴ denotes a hydrocarbon radical, preferably a C₁-C₁₈ alkylene radical or a C₁-C₁₈, and more preferably C₁-C₈, alkyleneoxy radical; Q⁻ is a halide ion, preferably chloride; r denotes an average statistical value from 2 to 20, preferably from 2 to 8; s denotes an average statistical value from 20 to 200, and preferably from 20 to 50. A preferred polymer of this class is available from Union Carbide under the name “UCAR SILICONE ALE 56.”

Other modified silicones or silicone copolymers are also useful herein. Examples of these include silicone-based quaternary ammonium compounds (Kennan quats) disclosed in U.S. Pat. Nos. 6,607,717 and 6,482,969; end-terminal quaternary siloxanes disclosed in German Pat. No. DE 10036533; silicone aminopolyalkyleneoxide block copolymers disclosed in U.S. Pat. Nos. 5,807,956 and 5,981,681; hydrophilic silicone emulsions disclosed in U.S. Pat. No. 6,207,782; silicone block copolymers with quaternary nitrogen groups disclosed in U.S. publications 20040048996A1 and 2004138400A1, and in WO02-10257 and WO02-10256; and polymers made up of one or more crosslinked rake or comb silicone copolymer segments disclosed in WO04-062634.

2. Polysorbate

The hair conditioning compositions of the present invention may contain a polysorbate, in view of adjusting rheology. Preferred polysorbate useful herein includes, for example, polysorbate-20, polysorbate-21, polysorbate-40, polysorbate-60, and mixtures thereof. Highly preferred is polysorbate-20.

The polysorbate can be contained in the composition at a level by weight of preferably from about 0.01% to about 5%, more preferably from about 0.05% to about 2%.

3. Polypropylene Glycol

Polypropylene glycol useful herein are those having a weight average molecular weight of from about 200 g/mol to about 100,000 g/mol, preferably from about 1,000 g/mol to about 60,000 g/mol. Without intending to be limited by theory, it is believed that the polypropylene glycol herein deposits onto, or is absorbed into hair to act as a moisturizer buffer, and/or provides one or more other desirable hair conditioning benefits.

The polypropylene glycol useful herein may be either water-soluble, water-insoluble, or may have a limited solubility in water, depending upon the degree of polymerization and whether other moieties are attached thereto. The desired solubility of the polypropylene glycol in water will depend in large part upon the form (e.g., leave-on, or rinse-off form) of the hair care composition. For example, a rinse-off hair care composition, it is preferred that the polypropylene glycol herein has a solubility in water at about 25° C. of less than about 1 g/100 g water, more preferably a solubility in water of less than about 0.5 g/100 g water, and even more preferably a solubility in water of less than about 0.1 g/100 g water.

The polypropylene glycol can be included in the hair conditioning compositions of the present invention at a level of, preferably from about 0.01% to about 10%, more preferably from about 0.05% to about 6%, still more preferably from about 0.1% to about 3% by weight of the composition.

4. Low Melting Point Oil

Low melting point oils useful herein are those having a melting point of less than about 25° C. The low melting point oil useful herein is selected from the group consisting of: hydrocarbon having from about 10 to about 40 carbon atoms; unsaturated fatty alcohols having from about 10 to about 30 carbon atoms such as oleyl alcohol; unsaturated fatty acids having from about 10 to about 30 carbon atoms; fatty acid derivatives; fatty alcohol derivatives; ester oils such as pentaerythritol ester oils, trimethylol ester oils, citrate ester oils, and glyceryl ester oils; poly α-olefin oils; and mixtures thereof. Preferred low melting point oils herein are selected from the group consisting of: ester oils such as pentaerythritol ester oils, trimethylol ester oils, citrate ester oils, and glyceryl ester oils; poly α-olefin oils; and mixtures thereof,

Particularly useful pentaerythritol ester oils and trimethylol ester oils herein include pentaerythritol tetraisostearate, pentaerythritol tetraoleate, trimethylolpropane triisostearate, trimethylolpropane trioleate, and mixtures thereof. Such compounds are available from Kokyo Alcohol with tradenames KAKPTI, KAKTTI, and Shin-nihon Rika with tradenames PTO, ENUJERUBU TP3SO.

Particularly useful citrate ester oils herein include triisocetyl citrate with tradename CITMOL 316 available from Bernel, triisostearyl citrate with tradename PELEMOL TISC available from Phoenix, and trioctyldodecyl citrate with tradename CITMOL 320 available from Bernel.

Particularly useful glyceryl ester oils herein include triisostearin with tradename SUN ESPOL G-318 available from Taiyo Kagaku, triolein with tradename CITHROL GTO available from Croda Surfactants Ltd., trilinolein with tradename EFADERMA-F available from Vevy, or tradename EFA-GLYCERIDES from Brooks.

Particularly useful poly α-olefin oils herein include polydecenes with tradenames PURESYN 6 having a number average molecular weight of about 500 and PURESYN 100 having a number average molecular weight of about 3000 and PURESYN 300 having a number average molecular weight of about 6000 available from Exxon Mobil Co.

5. Cationic Polymer

Cationic polymers useful herein are those having a weight average molecular weight of at least about 5,000, typically from about 10,000 to about 10 million, preferably from about 100,000 to about 2 million.

Suitable cationic polymers include, for example, copolymers of vinyl monomers having cationic amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, and vinyl pyrrolidone. Other suitable spacer monomers include vinyl esters, vinyl alcohol (made by hydrolysis of polyvinyl acetate), maleic anhydride, propylene glycol, and ethylene glycol. Other suitable cationic polymers useful herein include, for example, cationic celluloses, cationic starches, and cationic guar gums.

6. Polyethylene Glycol

Polyethylene glycol can also be used as an additional component. The polyethylene glycols useful herein that are especially preferred are PEG-2M wherein n has an average value of about 2,000 (PEG-2M is also known as Polyox WSR® N-10 from Union Carbide and as PEG-2,000); PEG-5M wherein n has an average value of about 5,000 (PEG-5M is also known as Polyox WSR® N-35 and as Polyox WSR® N-80, both from Union Carbide and as PEG-5,000 and Polyethylene Glycol 300,000); PEG-7M wherein n has an average value of about 7,000 (PEG-7M is also known as Polyox WSR® N-750 from Union Carbide); PEG-9M wherein n has an average value of about 9,000 (PEG-9M is also known as Polyox WSR® N-3333 from Union Carbide); and PEG-14M wherein n has an average value of about 14,000 (PEG-14M is also known as Polyox WSR® N-3000 from Union Carbide). As used herein “n” refers to the number of ethylene oxide units in the polymer.

### Method of Use

The hair conditioning compositions of the present invention are used in conventional ways to provide conditioning and other benefits. Such method of use depends upon the type of composition employed but generally involves application of an effective amount of the product to the hair or scalp, which may then be rinsed from the hair or scalp (as in the case of hair rinses) or allowed to remain on the hair or scalp (as in the case of gels, lotions, creams, and sprays). “Effective amount” means an amount sufficient enough to provide a dry conditioning benefit. In general, from about 1 g to about 50 g is applied to the hair or scalp.

The composition may be applied to wet or damp hair prior to drying of the hair. Typically, the composition is used after shampooing the hair. The composition is distributed throughout the hair or scalp, typically by rubbing or massaging the hair or scalp. After such compositions are applied to the hair, the hair is dried and styled in accordance with the preference of the user. In the alternative, the composition is applied to dry hair, and the hair is then combed or styled in accordance with the preference of the user.

### Product Forms

The hair conditioning compositions of the present invention can be in the form of rinse-off products or leave-on products (e.g., the compositions are applied to a user's skin and/or hair and a subsequent step of rinsing is omitted), can be opaque, and can be formulated in a wide variety of product forms, including but not limited to creams, gels, emulsions, mousses and sprays

### Non-Limiting Examples

The compositions illustrated in the following Examples exemplify specific embodiments of the compositions of the present invention, but are not intended to be limiting thereof. Other modifications can be undertaken by the skilled artisan without departing from the spirit and scope of this invention.

The compositions illustrated in the following Examples are prepared by conventional formulation and mixing methods, an example of which is described below. All exemplified amounts are listed as weight percents and exclude minor materials such as diluents, preservatives, color solutions, imagery ingredients, botanicals, and so forth, unless otherwise specified.

The compositions of the present invention are suitable for rinse-off products and leave-on products, and are particularly useful for making products in the form of a rinse off conditioner.

Compositions (wt %)

| Components | Ex. 1 | Ex. 2 | Ex. 3 | Ex. 4 |
|---|---|---|---|---|
| Nonionic silicone emulsion *1 | — | — | 2.4 | 4.2 |
| Cationic silicone emulsion *2 | 2.4 | 4.2 | — | — |
| Behenyl trimethyl ammonium | 3.38 | 2.25 | 3.38 | 2.25 |
| chloride *3 | | | | |
| Isopropyl alcohol | 0.899 | 0.598 | 0.899 | 0.598 |
| Cetyl alcohol *4 | 2.3 | 1.9 | 2.3 | 1.9 |
| Stearyl alcohol *5 | 4.2 | 4.6 | 4.2 | 4.6 |
| Polysorbate-20 *6 | — | — | 0.2 | — |
| PPG-34 *7 | 0.5 | — | — | — |
| Poly-α-olefin oil *8 | — | 0.5 | — | — |
| Benzyl alcohol | 0.4 | 0.4 | 0.4 | 0.4 |
| Methylchloroisothiazolinone/ | 0.0005 | 0.0005 | 0.0005 | 0.0005 |
| Methylisothiazolinone *9 | | | | |
| Perfume | 0.5 | 0.5 | 0.5 | 0.35 |
| NaOH | 0.014 | 0.014 | 0.014 | 0.014 |
| Panthenol *10 | 0.05 | 0.05 | — | 0.05 |
| Panthenyl ethyl ether *11 | 0.05 | 0.05 | — | 0.05 |
| Hydrolyzed collagen *12 | 0.01 | 0.01 | 0.01 | — |
| Vitamin E *13 | 0.01 | 0.01 | 0.01 | — |
| Octyl methoxycinnamate | 0.09 | 0.09 | 0.09 | — |
| Benzophenone-3 | 0.09 | 0.09 | 0.09 | — |
| Disodium EDTA | 0.127 | 0.127 | 0.127 | 0.127 |

| | |
|---|---|
| Deionized Water | q.s. to 100% |

Definitions of Components
*1 HMW 2220 Non-ionic Emulsion: 61 percent nonionic emulsion of a high molecular weight divinyldimethicone/dimethicone copolymer, available from Dow Corning Corp.
*2 Dow Corning 5-7069 cationic, available from Dow Corning Corp.
*3 Behenyl trimethyl ammonium chloride/Isopropyl alcohol: Genamin KDMP available from Clariant
*4 Cetyl alcohol: Konol series available from Shin Nihon Rika.
*5 Stearyl alcohol: Konol series available from Shin Nihon Rika.
*6 Polysorbate-20: Glycosperse L-20K available from Lonza Inc.
*7 PPG-34: New Pol PP-2000 available from Sanyo Kasei.
*8 Poly-α-olefin oil: Puresyn 100 available from Exxon Mobil
*9 Methylchloroisothiazolinone/Methylisothiazolinone: Kathon CG available from Rohm & Haas
*10 Panthenol: Available from Roche.
*11 Panthenyl ethyl ether: Available from Roche.
*12 Hydrolyzed collagen: Peptein 2000 available from Hormel.
*13 Vitamin E: Emix-d available from Eisai.

Prepare the hair conditioning compositions by any conventional method well known in the art. They are suitably made as follows:

Heat deionized water to 85° C. Mix cationic surfactants and high melting point fatty compounds into the water. Maintain the water at a temperature of about 85° C. until the components are homogenized and no solids are observed. Cool the mixture to about 55° C. and maintain at this temperature to form a gel matrix. Add the indicated silicone emulsion to the gel matrix. When included, add poly α-olefin oils, polypropylene glycols, silicones, and/or polysorbates to the gel matrix. Maintain the gel matrix at about 50° C. during this time with constant stirring to assure homogenization. When included, add other additional components such as perfumes and preservatives at this point also. After homogenization, cool to room temperature.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

All documents cited in the Background, Summary of the Invention, and Detailed Description of the Invention are, in relevant part, incorporated herein by reference; the citation of any document is not to be construed as an admission that it is prior art with respect to the present invention.

What is claimed is:

1. A hair conditioning composition comprising:
a) a silicone copolymer emulsion with an internal phase viscosity of greater than about 120×10⁶ mm²/sec; and
b) a gel matrix comprising:
i) a cationic surfactant;
ii) a high melting point fatty compound; and
iii) an aqueous carrier.

2. The hair conditioning composition of claim 1 wherein said silicone copolymer emulsion is present in an amount of from about 0.1% to about 15% by weight of the composition.

3. The hair conditioning composition of claim 1 wherein said silicone copolymer emulsion is present in an amount of from about 0.5% to about 10% by weight of the composition.

4. The hair conditioning composition of claim 1 wherein said silicone copolymer emulsion is present in an amount of from about 1% to about 8% by weight of the composition.

5. The hair conditioning composition of claim 1 wherein said silicone copolymer emulsion has an internal phase viscosity of greater than about 150×10⁶ mm²/sec.

6. The hair conditioning composition of claim 1 wherein said silicone copolymer emulsion has an average particle size of less than about 1 micron.

7. The hair conditioning composition of claim 1 wherein said silicone copolymer emulsion has an average particle size of less than about 0.7 microns.

8. The hair conditioning composition of claim 1 wherein said silicone copolymer comprises a silicone copolymer, at least one surfactant, and water.

9. The hair conditioning composition of claim 8 wherein said silicone copolymer results from the addition reaction of:
a) a polysiloxane with reactive groups on both termini;
b) at least one silicone compound or non-silicone compound comprising at least one or at most two groups capable of reacting with said polysiloxane; and
c) a metal containing catalyst.

10. The hair conditioning composition of claim 9 wherein said polysiloxane is represented by the following formula: 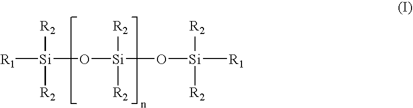
wherein R₁ is a group capable of reacting by chain addition reaction; R₂ is selected from the group consisting of alkyl, cycloalkyl, aryl, and alkylaryl; n is a whole number such that said polysiloxane has a viscosity of from about 1 mm²/sec to about 1×10⁶ mm²/sec.

11. The hair conditioning composition of claim 10 wherein said R₁ is selected from the group consisting of a hydrogen atom, an aliphatic group with ethylenic unsaturation, a hydroxyl group, and alkoxyl group, an acetoxyl group, an amino group, and an alkylamino group.

12. The hair conditioning composition of claim 10 wherein said R₁ is hydrogen.

13. The hair conditioning composition of claim 10 wherein said R₂ includes additional functional groups.

14. The hair conditioning composition of claim 13 wherein said additional functional groups are selected from the group consisting of ethers, hydroxyls, amines, carboxyls, thiols, esters, and sulfonates.

15. The hair conditioning composition of claim 10 wherein said R₂ includes less than about 10% on a mole percentage basis of said R₁ group.

16. The hair conditioning composition of claim 10 wherein said R₂ includes less than about 2% on a mole percentage basis of said R₁ group.

17. The hair conditioning composition of claim 10 wherein said R₂ is methyl.

18. The hair conditioning composition of claim 1 wherein said high melting point fatty compound is present in an amount of from about 0.1% to about 20% by weight of the composition.

19. The hair conditioning composition of claim 1 wherein said cationic surfactant is present in an amount of from about 0.1% to about 10% by weight of the composition.

20. The hair conditioning composition of claim 1 wherein said cationic surfactant has the following general formula: 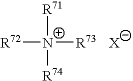
wherein at least one of said R⁷¹, R⁷², R⁷³ and R⁷⁴ is an aliphatic group of from about 16 to about 30 carbon atoms, and the remainder of said R⁷¹, R⁷², R⁷³ and R⁷⁴ are independently selected from the group consisting of a hydrogen, and an aliphatic group of from about 1 to about 22 carbon atoms;
wherein X⁻ is a salt-forming anion selected from the group consisting of halogen, acetate, citrate, lactate, glycolate, phosphate, nitrate, sulfonate, sulfate, glutamate, alkylsulfate, and alkyl sulfonate radicals.

21. The hair conditioning composition of claim 20 wherein the at least one of said R⁷¹, R⁷², R⁷³ and R⁷⁴ that is an aliphatic group of from about 16 to about 30 carbon atoms includes an aromatic, alkoxy, polyoxyalkylene, alkylamido, hydroxyalkyl, aryl or alkylaryl group having up to about 30 carbon atoms.

22. The hair conditioning composition of claim 20 wherein said cationic surfactant is an alkyltrimethylamine that includes an alkyl group having from about 16 to about 22 carbons.

23. The hair conditioning composition of claim 20 wherein said cationic surfactant is an alkylamidopropyldimethylamine that includes an alkyl group having from about 16 to about 22 carbons and is neutralized by lactic acid, glutamic acid, acetic acid, or a halide acid.

24. The hair conditioning composition of claim 1 further comprising an additional component selected from the group consisting of silicone, polysorbate, polypropylene glycol, low melting point oil, cationic polymer, and polyethylene glycol.

25. The hair conditioning composition of claim 1 wherein the composition is a leave-on product.

26. A hair conditioning composition comprising:
a) a first silicone compound comprising a silicone copolymer emulsion with an internal phase viscosity of greater than about 120×10⁶ mM²/sec;
b) a second silicone compound that is different than the first silicone compound; and
c) a gel matrix comprising:
i) a cationic surfactant;
ii) a high melting point fatty compound; and
iii) an aqueous carrier.

27. A method of providing improved conditioning benefits to hair and/or skin, said method comprising the step of applying to said hair and/or skin the conditioning composition of claim 1.

28. The method of claim 27 wherein the step of applying to said hair and/or skin the conditioning composition of claim 1 is not followed by a step of rinsing said hair and/or skin.
